(19) United States
(12) Patent Application Publication
(10) Pub. No.: US 2025/0360049 A1
(43) Pub. Date: Nov. 27, 2025

(54) The LFSS (Low Frequencies Sound Stimulation) Modular and Integrative Ergonomic Bedding KIT

(71) Applicant: TheSoundWell Vibro-Therapy, Boca Raton, FL (US)

(72) Inventor: Avigail BERG, Boca Raton, FL (US)

(21) Appl. No.: 18/670,180

(22) Filed: May 21, 2024

(51) Int. Cl. A61H 23/02; G16H 20/70

(52) U.S. Cl. CPC A61H 23/0236; G16H 20/70; A61H 2201/0107; A61H 2201/0146; A61H 2201/0157; A61H 2201/10; A61H 2201/1207; A61H 2201/1638; A61H 2201/1645; A61H 2201/165; A61H 2201/5002; A61H 2201/5012; A61H 2201/5035; A61H 2201/5058; A61H 2201/5097; A61H 2205/06; A61H 2205/10; A61H 2230/045; A61H 2230/10; A61H 2230/201; A61H 2230/505

(57) ABSTRACT

A kit adapted for low frequency sonic therapy consisting of a blanket, fitted topper, sleeve and pillow. These elements are fitted with transducers adapted to produce low frequency sound in the range of 30-120 Hz to provide a soft, gentle inner body massage. The blanket may in some cases be made especially heavy by means of weights. By combining the soothing qualities of a comfortable blanket, topper, sleeve and pillow with the therapeutic benefits of different frequencies, this invention aims to provide users with a unique and effective tool for improving their overall well-being-reducing mental, emotional, and physical stress, reducing insomnia and clearing the mind from negative thoughts. The particular sequence of tones produced by the transducers may be controlled by the user by means of an app running on a portable device. This enables users to explore the impact of different combinations of frequencies to their wellness, health and well-being.

### BACKGROUND OF THE INVENTION

The invention relates to the field of sonic therapy, and more specifically LFSS (low frequency Sound Stimulation) therapy, also known as Vibroacoustic Therapy. Traditional methods of achieving relaxation, deep sleep and musculoskeletal therapy often involve the use of external devices or substances, binaural audio, or audible music. Use of low sound frequencies sound waves for such purposes are known, but generally involve cumbersome hardware or imprecise techniques. For example JP5686362B2 “Sound therapy bed” presents an acoustic device adapted to increasing body temperature in an attempt to reduce the possibility of hypothermia, with the goal of preventing immune suppression and consequent disease that the authors feel may be related to hypothermia, including cancer, virus-borne illness, and heart disease. Thus, the acoustic therapy of the invention is directed entirely towards raising body temperature, by directing sound energy to the spinal cord and spine where the central nervous system, blood, and lymph collect, and to resonate the spinal cord and spine to increase body temperature by 1 to 2 degrees. The device uses a wide range of frequencies instead of concentrating on one particularly effective regime, and the acoustic source is embedded in the bed frame and not the bedding or blanket.

Likewise, US20150283019A1 “Tactile transducer treatment system” provides a foundation or base configured to support a mattress. The foundation is fitted with a transducer connected to a processing system adapted to provide a treatment regime. The treatment involves binaural audio and an isochrony tone adapted to produce a tactile response in the foundation. Such response has been found to be beneficial in the treatment of several patient conditions including autism, attention deficit disorder, attention deficit hyperactivity disorder, post-traumatic stress disorder, bed wetting, and/or a variety of sleep disorders. While the use of binaural sound for therapy may be a promising direction, LFSS is a regime not explored by this application.

JP2008000222A “Quiet sleep support system, quiet sleep support program and its recording medium” uses low-frequency vibration by means of rhythms selected or preferred by users. A sleeping state identification sensor is attached to bedding or an armchair, adapted for detecting the sleeping state of the user, with control mechanisms adapted to provide an ‘optimal’ signal depending upon the sleeping state of the user. However, while this system does use low frequency vibration, the hardware setup allows only for vibration from above the body. Furthermore, the use of sensors to determine the user's sleep state is a more complex approach than necessary, given that states of deep relaxation may be reached simply by programmed LFSS regimens without feedback, as will be explained in the following.

Thus, there remains a need for a convenient, non-intrusive, and effective solution that can enhance relaxation, reduce mental, emotional, and physical stress daily and promote deep sleep without relying on these techniques.

### SUMMARY OF THE INVENTION

The LFSS Modular and Integrative Ergonomic Bedding KIT includes four modular and integrated pieces, which can be used together or separately:
1. Soft Snuggling Sound Blanket, AKA-LFSS—Blanket (FIGS. 5-8)
2. Sound Bath Topper, AKA-LFSS—Bed Topper (FIG. 9-10)
3. Sonic Harmonic sleeve, AKA LFSS—Sleeve (FIG. 13)
4. Sonic Cuddly Pillow, AKA-LFSS Cuddly Pillow (FIG. 4)

Each element of the Kit acts with its own LFSS capabilities and is connected to the dedicated Low Sound Frequency App. The diagram of FIG. 18 shows a cloud-based server in communication with the mobile app of the invention, which will generally be running on a user's smartphone, tablet, or other mobile device. There is also an option to download the app locally to a mobile device and run the low sound frequencies-offline.

The mobile app in turn controls the transducers of the various articles of the kit (topper, blanket, pillow, and sleeve).

The transducers will generally be controlled by a remote app of the invention, running a wireless communications protocol such as Bluetooth (or can be operated by a cable to the amplifier). For example, a tablet (801 of FIG. 8) running the app of the invention, may be connected wirelessly to the blanket of the invention. The app will generally run locally on a user's mobile device. This app connects in some steps to a server which is, in one embodiment, adapted to allow for registration and for registered users to download the app of the invention. The app then runs locally on the user's mobile device (smartphone, tablet, etc.) Steps involving authentication, registration, database communications, user profile storage and so on can be carried out on a server of the invention. The communication between mobile app and transducer may be one-way, with the app simply controlling the transducer via the amplifier to produce tones of various frequency and duration.

The invention is designed as a kit having several units—topper, blanket, pillow, and sleeve. Users can purchase each unit independently; however, the kit enables various advantages—such as mobility of the blanket (to other room), The sleeve activates harmonic vibrations in a beanbag or a sofa, the pillow that may be used on the go, and the availability of a powerful whole body soundbath achieved when more than one unit of the invention is used simultaneously.

The LFSS—Blanket is a specially designed blanket with integrated low frequency sound transducers, strategically placed throughout its structure. The transducers emit low-frequency sound waves in the range between 30 hz and 120 Hz. These frequencies have been scientifically proven to induce relaxation, reduce mental, emotional, and physical stress, and reduce insomnia. A user sleeps under this blanket such that sound is transmitted to the user's torso and/or limbs from above.

The Bed Topper is a bedsheet or fitted sheet similarly integrated with low frequency sound transducers, such that sound is transmitted to the user's torso and/or limbs from below.

The Sonic Harmonic Sleeve is a tube or sleeve fitted with low-frequency transducers. The sleeve may be placed on a particular area of the body such as leg or arm and can also activate vibrations in other furniture such as sofa or bean bag.

The Cuddly Pillow is a pillow similarly outfitted with low-frequency sound transducers, adapted to transmit low frequency sound to the user's body parts as s/he hugs it.

### BRIEF DESCRIPTION OF DRAWINGS

FIG. 1 shows a view of an amplifier suitable for use with the invention.

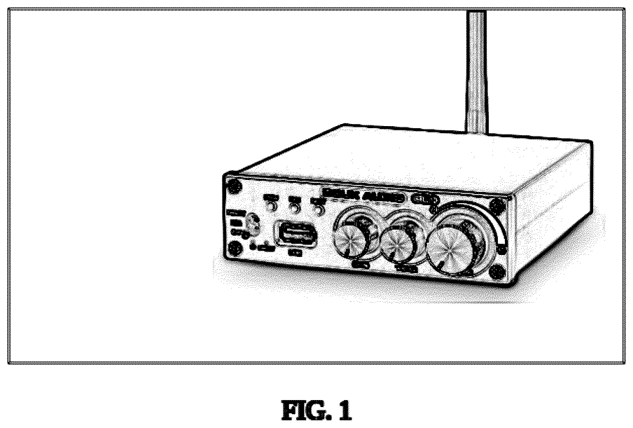

FIG. 2 shows a transducer suitable for use with the invention.

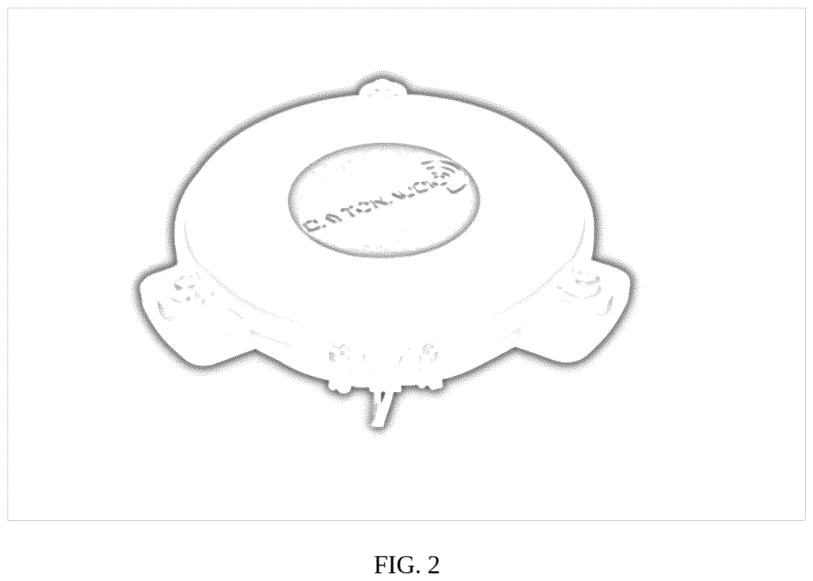

FIG. 3 shows a rechargeable, portable Bluetooth™ amplifier suitable for use with the invention.

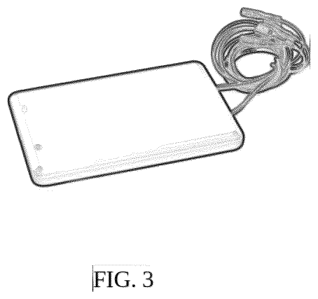

FIG. 4 shows a view of the pillow of the invention, with transducers embedded therein.

FIG. 5 shows a view of the blanket of the invention, with transducers embedded therein.

FIG. 6 shows a closeup view of the blanket of the invention, with zipper.

FIG. 7 shows a person sleeping between the topper and blanket of the invention.

FIG. 8 shows a portable device connected to a blanket of the invention.

FIG. 9 shows a view of the topper of the invention with the positioning of the transducers built in.

FIG. 10 shows a schematic top view of the positioning of the transducers of the topper.

FIG. 11 shows a view of the blanket of the invention with the positioning of the transducers built in, superimposed over the positions of the topper transducers.

FIG. 12 shows perspective views of the topper of the invention with possible transducer placements indicated.

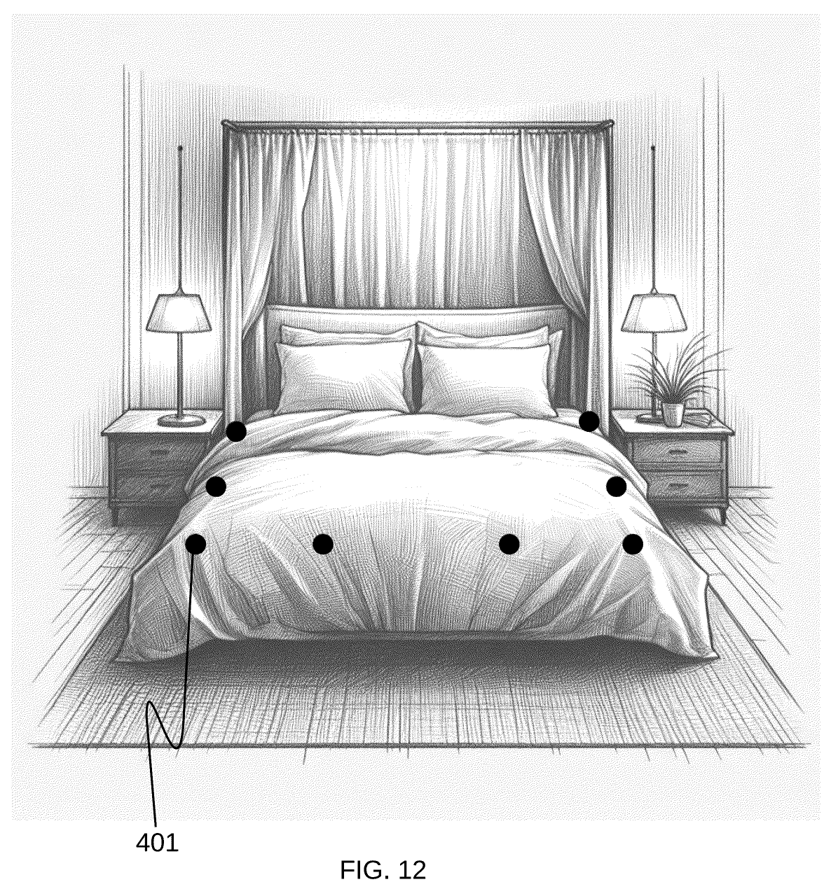

FIG. 13 shows the sleeve of the invention with possible locations of the transducers shown.

FIG. 14 shows a possible embodiment of a sleeve of the invention.

FIG. 15 shows a possible flow chart for operation of the app of the invention.

FIG. 16 shows a possible planning chart for users to map out their plans for LFSS using the invention.

FIG. 17 shows a ‘feedback chart’ adapted to let users record their impressions of various LFSS programs for planning future use.

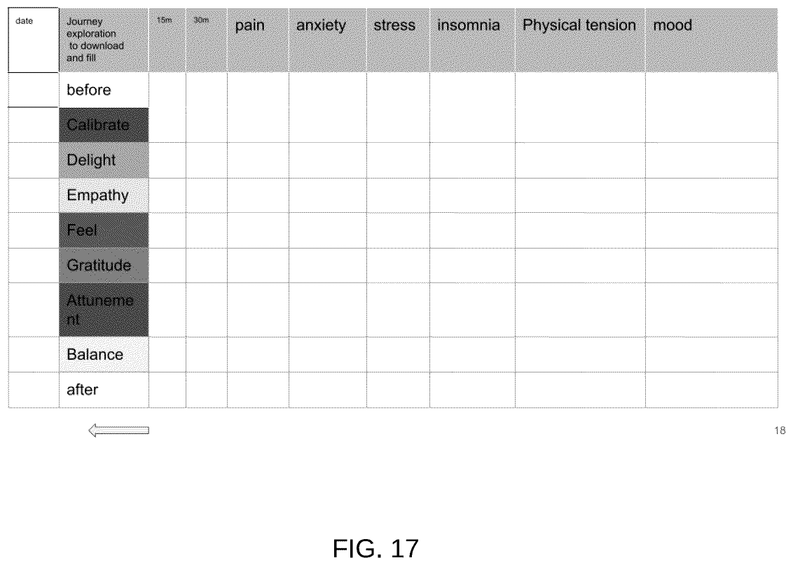

FIG. 18 shows a diagram implementing one possible setup for operation of the inventive method.

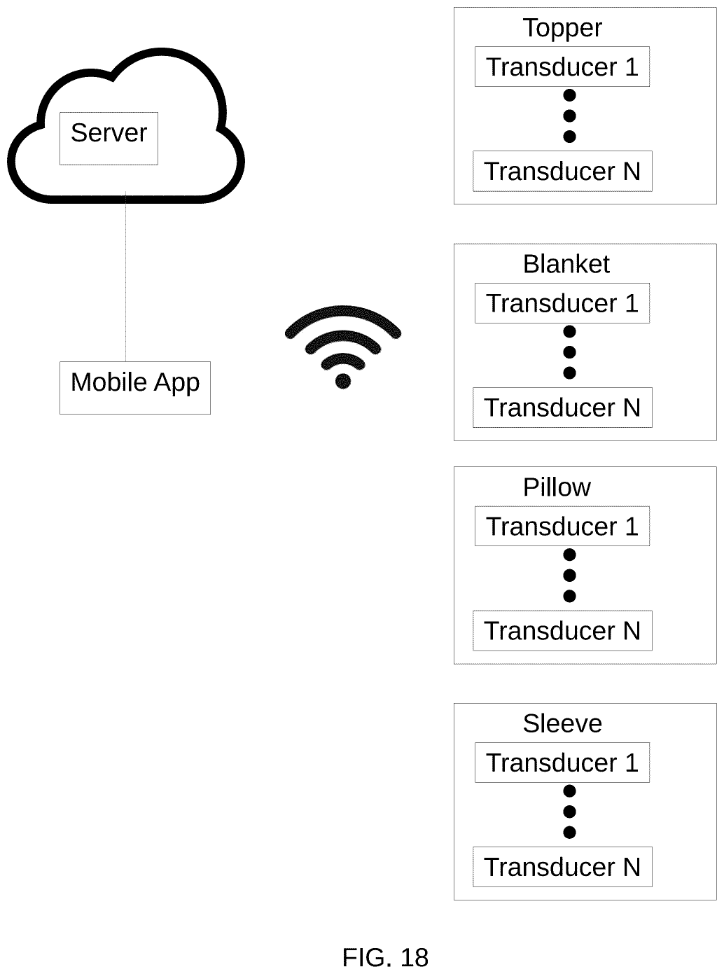

FIG. 19 shows a flowchart implementing one possible embodiment of the invention.

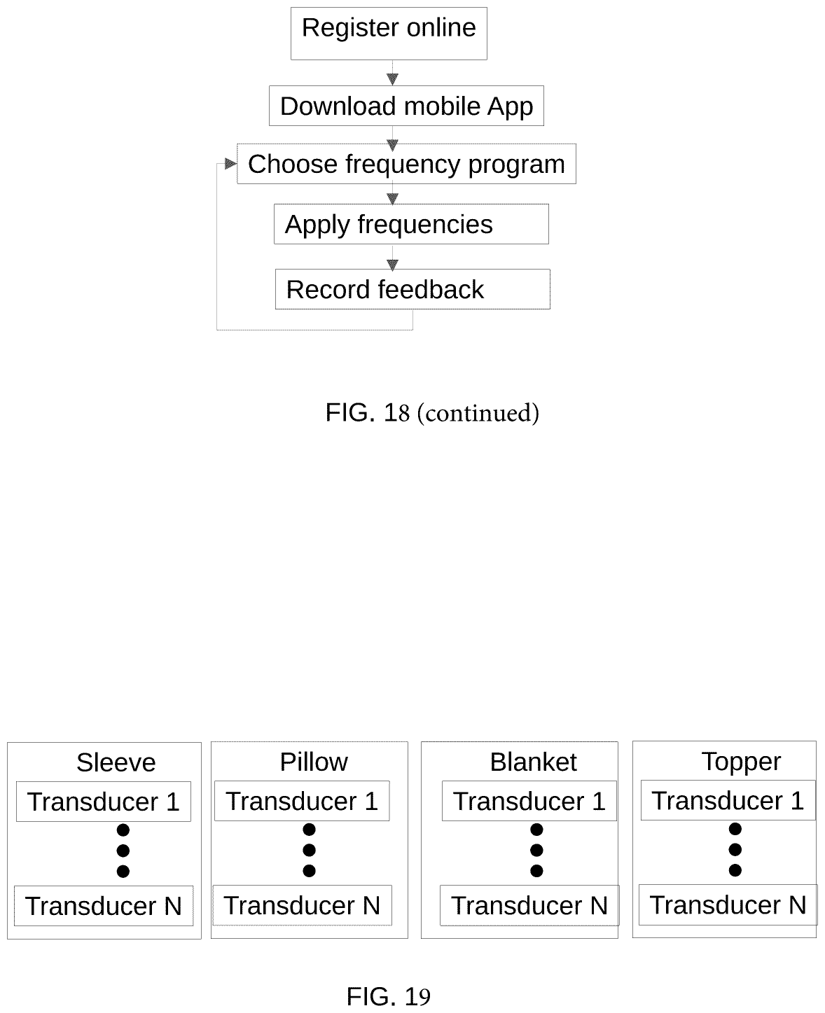

### DETAILED DESCRIPTION OF THE INVENTION

The following detailed description provides a comprehensive overview of the key components and features of the invention. The invention comprises a unique combination of textile materials used to form various objects, low sound frequency transducers, and control program (in one embodiment taking the form of an app) adapted to provide users with a novel and effective means of relaxation, reduction of mental, emotional, and physical stress and improved sleep quality. Generally, the invention has between two to four transducer-containing parts which can be used together or separately; a blanket; a bed topper; a sleeve and a hugging pillow.

### Blanket

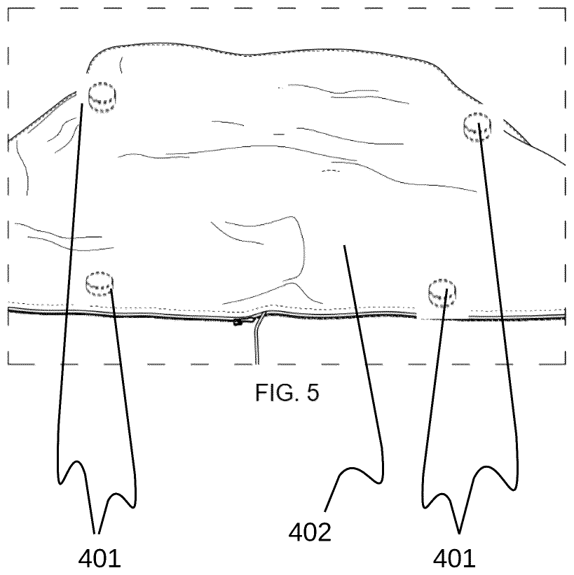

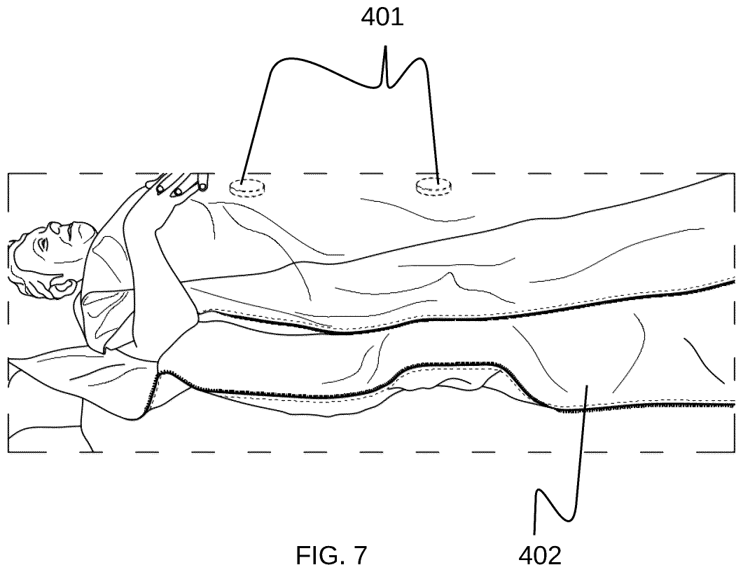

In one embodiment, the device comprises a heavy blanket such as that shown in FIGS. 5-8. The device is further provided with power supply, amplifier, and low sound frequency transducers adapted to produce a number of sound compositions tones in the frequency range of 30-120 Hz. The transducers 401 in the blanket 402 are embedded within the blanket itself, simplifying the use thereof, it presents a standard blanket in all respects except possibly for its electrical connections. The power supply to the amplifier may be accomplished by means of a battery, low-voltage dc power supply, or inductive coupler. In the case of use of a battery, the blanket is made ‘mobile’ and can be carried about and used without regard for the electrical connection.

The blanket may in some cases be made especially heavy by means of weights. As has been shown, the use of deep pressure bodily stimulation produces a calming effect in both humans and animals.¹ It has been reported that “weighted blankets may benefit people with anxiety, autism, and insomnia, among other conditions.”² ¹ https://www.bancroft.org/resources/the-calming-effect-temple-grandin/² https://www.healthline.com/health/anxiety/do-weighted-blankets-work

By combining the soothing qualities of a comfortable blanket with the therapeutic benefits of low sound frequency that are converted into soft and gentle inner body massage by the transducers, this invention aims to provide users with a unique and effective tool for improving their overall well-being. Unlike the methods of prior art, the transducers in the current invention are located within the blanket itself, allowing for the user to be ‘cocooned’ by the sound produced by the blanket.

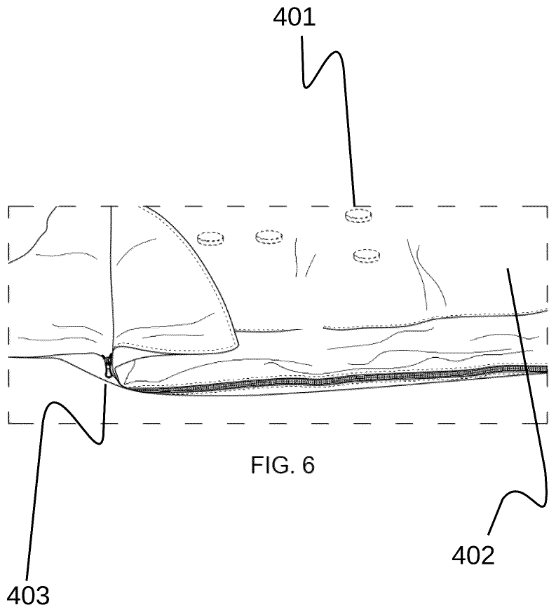

Users may lie under the blanket, on top of it or wrap it around themselves as a sleeping bag, allowing for effectively surrounding the user with sound sources and thus enabling whole-body effects not possible when the sound sources are only located on one side of the body. In further embodiments, fastenings or closures such as snaps or zippers (see FIG. 6, 403) may be provided allowing a single blanket or comforter to be used as a sleeping bag, by folding it over itself lengthwise and fastening the long side closed to form a cocoon.

### Bed Topper (aka Fastened Cover/Pad)

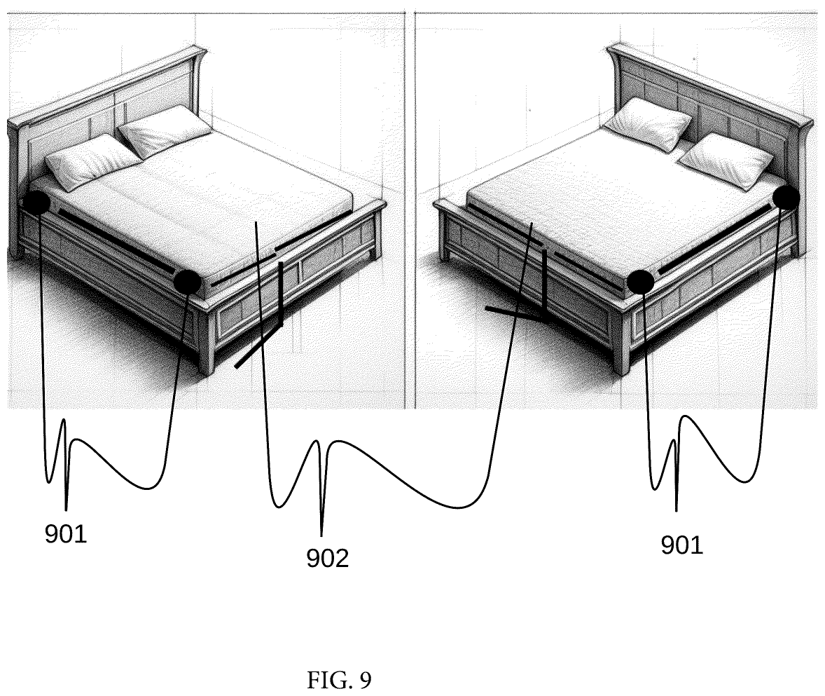

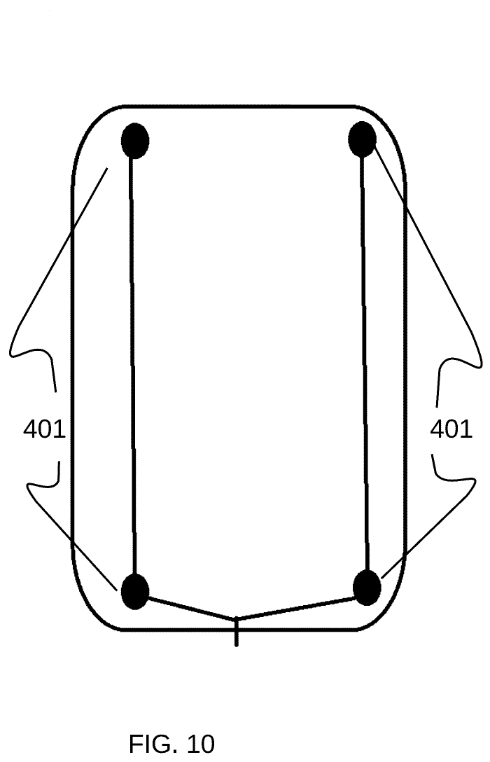

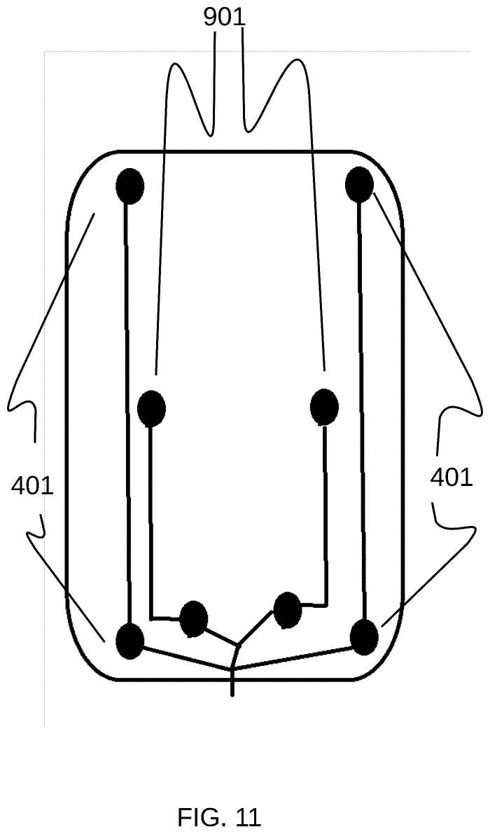

In a similar fashion to the blanket, a bed sheet or ‘topper’ is provided, as shown for example in FIG. 9 (902). The topper is adapted to fit snugly around a mattress. This topper 902 is likewise fitted with low sound frequency transducers 901 adapted to produce a number of harmonic low sound compositions in the frequency range of 30-120 Hz. The topper is thus adapted to produce sound and vibration from below the user's body, who lies upon it when sleeping or undergoing therapy. As with the blanket, the transducers in the topper are built in. The topper in most ways is identical to a standard bed topper or fitted sheet (with elastic bands helping to keep the sheet fitted to the bed) except for the sound cables, which will be connected to external amplifier. Since the sonic ergonomic kit is modular, users may decide to use only the bed topper which covers the mattress, and select the desired frequency from the app. The complementary positioning of the transducers in the topper and in the blanket enables a powerful immersive soundbath effect on user's body. This arrangement is shown in FIGS. 10 and 11 where the transducers of the blanket 401 are shown in FIG. 10, and the positions of topper 901 are shown in FIG. 11, superimposed upon the blanket transducer positions.

### Sonic Harmonic Sleeve

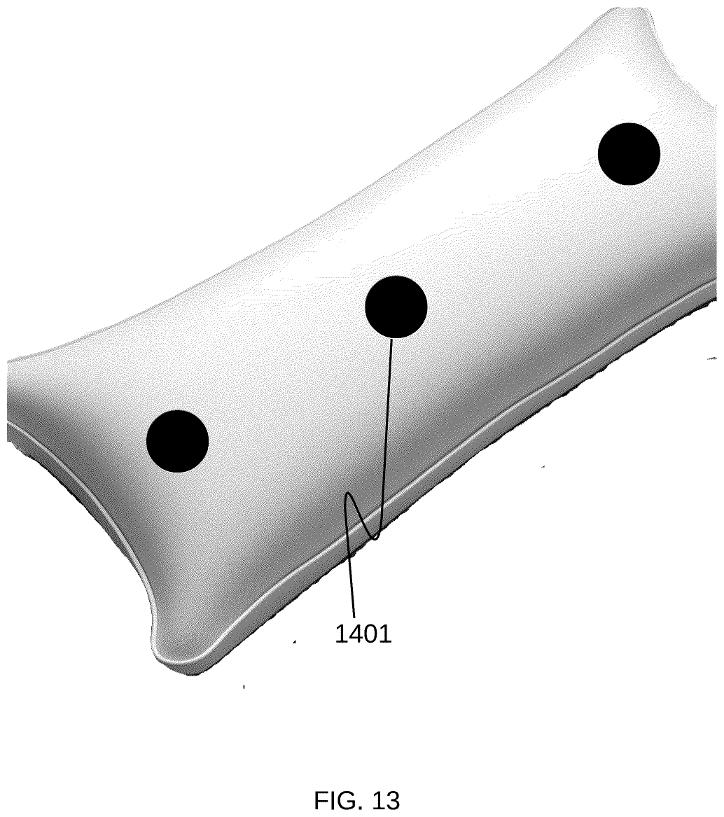

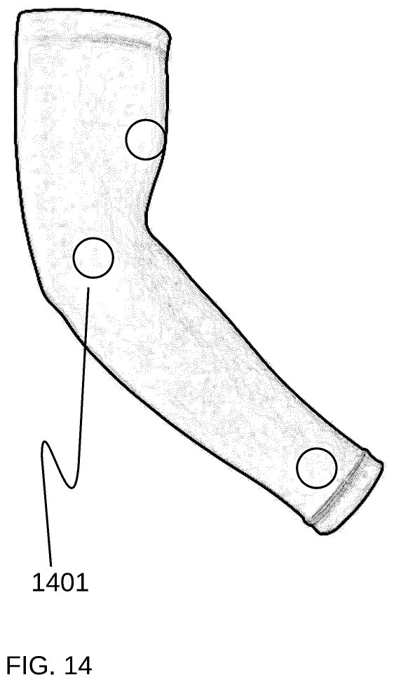

A further optional element of the kit is a sleeve-like device such as that shown in FIGS. 13 and 14, that fits around a user's arm or leg and which is also fitted with low sound frequency transducers 1401 adapted to produce tones in the frequency range of 30-120 Hz. The sleeve is thus adapted to produce sound and vibration around the user's focused location. The purpose of the sleeve is to allow a user to use vibratory therapy in a number of new contexts. First of all, the sleeve as mentioned may be used for directly affecting the arm of leg encased thereby. Other uses however are possible. For example, the sleeve may be placed under pillows of a sofa or inside a beanbag, turning each of these furniture items into sonic ergonomic vibrating items. As with the blanket and the topper the transducers 1401 are located in the sleeve itself. Like the other kit elements, the sleeve may have an amplifier built in, with provision for use of a rechargeable battery or inductive or wired power source or can use an external amplifier connected to the wall electricity output. The sleeve enables users to add more sonic therapy elements to additional rooms in the home (living room, porch, children's room, etc). The Sleeve may be for instance 30″ length by 12″ diameter by 2″ thickness and may have between 2-4 transducers. The cover of the sleeve is made of a comfortable and flexible material, such as vinyl leather.

### Pillow

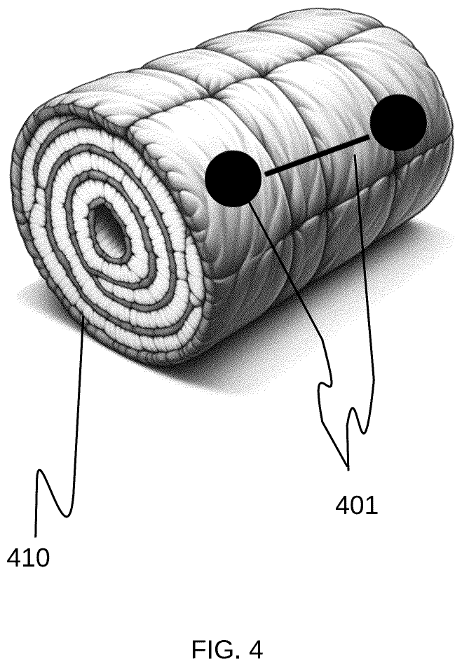

As with the blanket and the topper, the pillow is provided as a further element of the inventive set of sonic ergonomic modular and integrative bedding kit. The pillow, shown for example in FIG. 4 (element 410) is likewise fitted with low sound frequency transducers 401 adapted to produce a number of sound compositions in the frequency range of 30-120 Hz. The pillow is thus adapted to produce sound and vibration from below the user's body, who lies upon or hugs it when sleeping or undergoing therapy. As with the blanket and the topper the transducers are located in the pillow itself. The pillow has an amplifier built in, with provision for use of a rechargeable battery. Due to this built-in power source, the pillow can be taken on the go anywhere. The pillow is very soft and intended to act as emotional support while user can hug it while resting or sleeping anywhere.

The pillow is made from very soft and gentle fabric. It is very light to carry. Its intention is to hug it during sleep, or on the go to reduce stress, anxiety, fatigue and mild pain.

### External Speaker

The kit may further include one or more optional portable low-frequency speakers that, like the other kit components, that wirelessly connects to the app and allows users to enjoy low-frequency sonic therapy sessions in various settings, including outdoors, during travel, and possibly underwater in case of soundproof speakers. By using external speakers, the ‘surround’ effect of the device may be amplified, for example by placing one speaker in each corner of a room in which one is resting.

Some key components and provision of the blanket, topper, pillow, and sleeve of the invention include:
1. Material: The blanket and other kit elements are crafted from a blend of soft, breathable, and hypoallergenic materials, ensuring maximum comfort for users. The choice of materials aims to create a cozy and inviting surface for users to rest upon or under, while also allowing for effective transmission of the harmonic low sound vibrations. Generally, the kit pieces are made of materials that enhance the transmission and absorption of low-frequency sound waves, maximizing the therapeutic effect.
2. Positioning the low sound frequency transducers: Embedded within the edges layers of the blanket and other kit elements, a number of transducers are strategically positioned, for example to cover the entire surface area of the blanket, ensuring an even distribution of the sonic waves. The speakers are securely integrated into the fabric of the various kit elements to maintain the blanket's flexibility and comfort.
3. The app low sound frequency library: In one embodiment, users receive a special code to download a set of low sound frequency compositions. These consist of low sound frequency compositions or sequences, such as C1-C2, D1-D2, E1-E2, F1-F2, G1-G2, B1-B2 based on 432 Hz tuning A where C1 for instance refers to 32.11 Hz, C2 refers to 64.22 Hz, and so on. The tuning of the scale may be for example the tempered scale, natural (whole-number ratio) scale, or any other tuning found to be useful. Although the app has a default of 432 hz, users have the option to make changes of tuning.

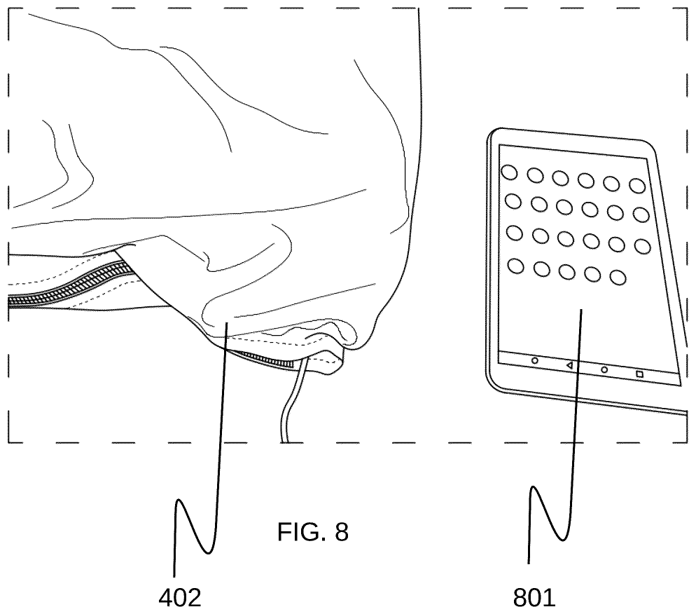

A sequence of tones or chords is referred to as a composition. The compositions are downloaded, entered, or otherwise input to a user's mobile device, as shown in FIG. 8. Without being bound by theory, the tones and/or compositions target different body parts and/or energy centers known as chakras, as described in Eastern medicine. It is suggested that the frequencies help the flow of energy in the body, preventing or opening blockages that may be causing or eventually lead to illnesses. Users can adjust the compositions, including intensity and frequency of individual tones they wish to use for fixed periods between 15 minutes, 30 minutes or stream it in a loop. The app enables users to create a plan of streaming different frequencies one after the other. The app is an integral part of the kit of the invention. The app enables exploration of different programs of self-wellbeing and care, in the comfort of the users bed, home, and on the go.
4. Amplifier: The blanket features an intuitive control method using wireless connectivity such as Bluetooth™ or by using cables which connects to a dedicated amplifier. The amplifier may be internal in the lower blanket edge or external. An associated tablet (e.g. 801 of FIG. 8) which contains the app is optional to be part of the system. The app can run the Topper and the blanket simultaneously, or if desired other combinations of the topper, blanket, sleeve, and pillow can be used simultaneously.
5. Power Source: The blanket is designed to be powered either by a rechargeable or replaceable battery system, or by means of direct connection to DC power derived from the mains, or by means of indirect or wireless power transfer such as by inductive coupling. This allows for a variety of connection options, making the blanket suitable for various settings, including bedrooms, lounges, or even outdoor spaces.
6. User Safety Features: The inventive device incorporates safety features to ensure user well-being and protection. These features may include automatic shut-off mechanisms, overheat protection, and compliance with relevant safety standards to guarantee a secure and enjoyable user experience.
7. Maintenance and Cleaning: The materials used in the blanket are selected to facilitate easy maintenance and cleaning. The blanket may be designed to be machine washable or have removable components for periodic cleaning, ensuring hygiene and longevity. In addition, blanket cases are offered to be purchased as part of the product components.

The LFSS (Low Frequencies Sound Stimulation) Modular and Integrative Ergonomic Bedding KIT has a number of advantages including flexibility of usage, which is facilitated by the following:
a. All components (blanket, topper, sleeve, pillow) are suitable for night-time use, in bed.
b. Several components may be used both in bed, and in the living room (blanket, sleeve, pillow).
c. The pillow and sleeve (and possibly, the blanket) can be used on the go (in a car, on vacation, etc.).
d. All components use the same app that can be downloaded to the user's phone or as part of a dedicated tablet.

The features of the LFSS in the topper are similar to the blanket. However, it may differ in the following respects:
e. Its material.
f. The transducers positions are placed in the 4 edges of the topper, which complement the 4 transducers in the blanket.
g. By its nature, a topper is stable (fitted to a bed) and does not enable mobility like the blanket.

The frequencies being employed by the device are preferably based on 432 hz tuning as opposed to the more common 440 Hz tuning. This seemingly small difference of 8 Hz has sparked debate and discussion in the music world for decades, but in practice this change translates to a slight decrease in pitch, with some claiming that 432 Hz sounds is “warmer,” “more natural,” or “more pleasing” than 440 Hz. Some also associate 432 Hz with improved well-being, relaxation, and spiritual connection, albeit without the basis of strong scientific evidence.

It is within the provision of the invention that the user may define their own tuning, using 432 Hz, 440 Hz, or any other tuning by means of settings in the app of the invention.

As mentioned, an important part of the invention is allowing the user to control which frequencies are being used and their progression. In this way, a user may be able to design a program of tones or frequencies that induce (for instance) a state of relaxation for an initial 1.5 hours, based on different frequencies that corelate to different body parts (related to the chakras) and then can go on to a state of deep sleep in a loop. A feedback worksheet is available for users to write down their subjective impressions of the quality of mood or mental state reached by each of the different frequencies of the device. By means of this feedback, users can program a sequence that is most effective for them, for example in terms of reducing pain, stress, insomnia, anxiety, or other woes. By programming an effective sequence, the user in essence designs an ‘Haptic, Tactile somatic journey’.

It is within the provision of the invention that harmonics of the fundamental frequency being used can be played along with the fundamental. For example, the octave may be played, or any set of chords may be played. It is also within provision of the invention that audio data from any streaming source may be played through the transducers of the invention, such as audio from YouTube channels, audio from streaming services, or audio from home stereo system.

Without being bound by theory, a provision of the invention is that it be used in conjunction with knowledge from Eastern medicine, in particular the theory of the Chakras and their associated locations, colors, and frequencies. The seven chakras may be addressed by means of seven frequencies of the inventions, which are intended to unblock or otherwise activate these chakras and their corresponding energetic flows as theorized in various forms of Eastern medicine. The chakras correlate in perception with acupuncture and meridians that are more and more accepted within western medicine.

It is envisioned that the blanket, the topper, sleeve, and pillow of the invention, in conjunction with the programmable sequence allowed for by the app of the invention, will constitute a ‘wellness platform’ that users will return to consistently due to the benefits experienced.

### App

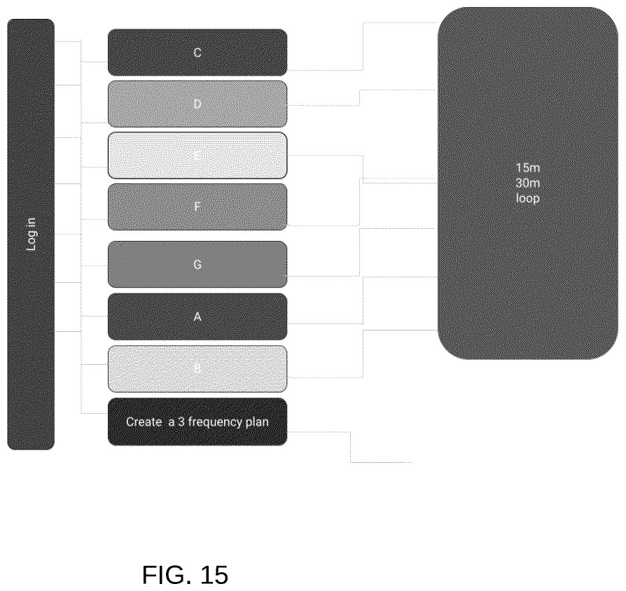

The app of the invention is adapted to allow the user to select various tones and sequences for various types of therapy. As seen in FIG. 14, steps of login, creating plans of sequences of frequencies, and playing these frequencies in various loops, is provided for in the app of the invention. FIG. 15 shows a table a user can use to select the desired frequencies for various time periods, in this case 15 or 30 minutes.

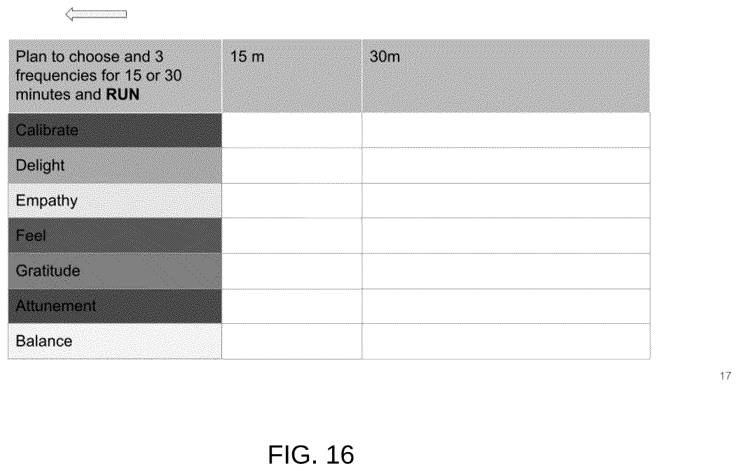

FIG. 16 shows a feedback sheet the user can employ in order to record their own idiosyncratic responses to the various frequencies of the invention that they have used. By recording this feedback, a user can select those frequencies and sequences that prove most effective for their own particular needs. By means of the app control, the user can thus design their own therapy, possibly changing the tones and sequences when their own needs and/or responses change.

The app includes a database of pre-programmed therapeutic sequences based on user feedback and expert recommendations, offering users a curated selection of wellness routines.

### Biofeedback

In some embodiments of the invention, sensors are integrated into the kit pieces and/or onto the user's body. The sensors measure physiological parameters of the user and may be used to manually or automatically adjust the compositions of low-frequency sound waves based on real-time feedback. Sensors may include heart rate, oxygen saturation, blood pressure, body temperature, EEG data such as dominant frequencies, and so on. In such cases, the app may also allow for measurement and recording of biofeedback relating to a user's mental, emotional, and physical well-being, enabling the user to improve the operation of said sonic therapy system towards attaining various states of well-being.

The low-frequency sonic therapy of the invention is specifically tuned to reduce mental stress, emotional stress, physical stress, insomnia, and negative thoughts.

1. A sonic therapy system consisting of:
a. four modular and integrated pieces each provided with low frequency transducers adapted to produce tones in the frequency range of 30-120 Hz to produce sound and vibration, a low frequency sound amplifier powering said transducers, and a power supply, said pieces being:
i. a blanket.
ii. a fitted topper adapted to fit a mattress.
iii. an ergonomic sleeve.
iv. a pillow.
b. an app adapted to control said amplifiers to produce low-frequency sounds from said transducers, thereby creating low sound frequency compositions.
whereby low frequency sonic therapy may be carried out while a user sleeps.

2. The system of claim 1 further providing a sleeve adapted to fit around a user's arm or leg, said sleeve being fitted with low sound frequency transducers adapted to produce tones in the frequency range of 30-120 Hz to produce sound and vibration around said user's arm.

3. The system of claim 1, further comprising sensors integrated into said pieces and/or onto a user's body, wherein said sensors measure physiological parameters of the user and adjust the frequency and intensity of the low-frequency sound waves based on real-time feedback.

4. The system of claim 1, wherein said app allows for measurement and recording of biofeedback relating to a user's mental, emotional, and physical well-being, enabling said user to improve the operation of said sonic therapy system towards attaining said well-being, track progress, and receive recommendations for optimizing their sonic therapy regimen.

5. The system of claim 1, wherein said low-frequency sonic therapy is specifically tuned to reduce mental stress, emotional stress, physical stress, insomnia, and negative thoughts.

6. The system of claim 1 wherein said power supply is selected from the list consisting of one or more of: battery; low-voltage dc power supply; inductive coupler.

7. The system of claim 1, wherein said pieces are made of materials that enhance the transmission and absorption of low-frequency sound waves, maximizing the therapeutic effect.

8. The system of claim 1, wherein the app includes a database of pre-programmed therapeutic sequences based on user feedback and expert recommendations, offering users a curated selection of wellness routines.

9. The system of claim 1, further comprising a portable speaker system that wirelessly connects to said pieces, allowing users to enjoy low-frequency sonic therapy sessions in various settings, including outdoors or during travel.

10. A method for production of desired states of relaxation in a user, consisting of:
a. surrounding said user with sources of low frequency sound waves.
b. controlling the duration, intensity, and sequence of said low frequency sound waves by means of an app used by said user.
c. personal journey of self-help documentation of the planned combinations of frequencies and their impact on mental, emotional and physical systems.

11. The method of claim 10 further providing a sleeve adapted to fit around a user's arm or leg, said sleeve being fitted with low sound frequency transducers adapted to produce tones in the frequency range of 30-120 Hz to produce sound and vibration around said user's arm.

12. The method of claim 10, further comprising sensors integrated into said pieces and/or onto a user's body, wherein said sensors measure physiological parameters of the user and adjust the frequency and intensity of the low-frequency sound waves based on real-time feedback.

13. The method of claim 10, wherein said app allows for measurement and recording of biofeedback relating to a user's mental, emotional, and physical well-being, enabling said user to improve the operation of said sonic therapy system towards attaining said well-being, track progress, and receive recommendations for optimizing their sonic therapy regimen.

14. The method of claim 10, wherein said low-frequency sonic therapy is specifically tuned to reduce mental stress, emotional stress, physical stress, insomnia, and negative thoughts.

15. The method of claim 10 wherein said power supply is selected from the list consisting of one or more of: battery; low-voltage dc power supply; inductive coupler.

16. The method of claim 10, wherein said pieces are made of materials that enhance the transmission and absorption of low-frequency sound waves, maximizing the therapeutic effect.

17. The method of claim 10, wherein the app includes a database of pre-programmed therapeutic sequences based on user feedback and expert recommendations, offering users a curated selection of wellness routines.

18. The method of claim 10, further comprising a portable speaker system that wirelessly connects to said pieces, allowing users to enjoy low-frequency sonic therapy sessions in various settings, including outdoors or during travel.
